(19) United States
(12) Patent Application Publication
(10) Pub. No.: US 2005/0136056 A1
(43) Pub. Date: Jun. 23, 2005

(54) Pharmaceutical composition for the treatment of thrombocytopenia

(76) Inventors: Shunsuke Kageyama, Tokyo (JP); Ken Hirose, Kawasaki-shi (JP); Hiroshi Yamamoto, Kawasaki-shi (JP); Makoto Shiozaki, Tokyo (JP); Junko Matsushita, Kawasaki-shi (JP)

Correspondence Address: CERMAK & KENEALY LLP;ACS LLC, 515 EAST BRADDOCK ROAD, SUITE B, ALEXANDRIA, VA, 22314, US

(21) Appl. No.: 11/044,198

(22) Filed: Jan. 28, 2005

Related U.S. Application Data
(63) Continuation of application No. PCT/JP03/09503, filed on Jul. 25, 2003.

(30) Foreign Application Priority Data
Jul. 29, 2002 (JP) 2002-219173

(51) Int. Cl. A61K 39/395

(52) U.S. Cl. 424/145.1

(57) ABSTRACT

The present invention provides a drug for the treatment of thrombocytopenia caused by hepatic failure, preferably a drug with few adverse drug reactions. A substance that inhibits binding between glycoprotein Ib (GPIb) and von Willebrand factor (vWF), for example, anti-GPIb antibody or anti-vWF antibody that inhibits binding between GPIb and vWF is an active ingredient of the drug for the treatment of thrombocytopenia.

This application claims priority under 35 U.S.C. §120 to PCT/JP2003/009503 as a continuation.

### TECHNICAL FIELD

The present invention relates to a drug used for the treatment of thrombocytopenia having a substance that inhibits binding between glycoprotein Ib and von Willebrand factor as an active ingredient.

### BACKGROUND ART

Thrombocytopenia is a condition in which the number of platelets per unit volume of peripheral blood is lower than normal. Specifically, thrombocytopenia refers to a decrease in the platelet count to 100,000/μL or lower compared to the normal platelet count, which generally ranges from 150,000 to 350,000/μL. Petechial bleeding and purpura are most frequently observed as initial symptoms, followed by nasal bleeding, gingival bleeding, and the like. Early diagnosis and early treatment are important also for the prevention of progress to more serious symptoms, such as cerebral bleeding. The following four factors (1) failed platelet production, (2) abnormal platelet distribution, (3) increased platelet destruction, and (4) increased platelet consumption are known as the mechanism of thrombocytopenia, however, its pathological features and causes vary widely. Specially, thrombocytopenia attributable to hepatic failure has been considered to be clinically problematic for a long time as one of the complications affecting patient prognosis. Some types of von Willebrand disease are rare diseases that exhibit thrombocytopenic symptoms and a bleeding tendency as a result of increased platelet consumption in the body due to congenital qualitative disorders of glycoprotein Ib and von Willebrand factor.

Hepatic failure is a condition in which the functional hepatic parenchyma decreases due to some cause so that hepatic functions cannot be maintained, and it is classified into acute (mainly, fulminant hepatitis) and chronic (mainly, hepatic cirrhosis) types. Acute hepatic failure is generally a condition in which a significant hepatic disorder suddenly occurs in an individual who has no previous history of apparent hepatic disease, resulting in hepatic failure or a drastic aggravation of chronic hepatic failure. Viral, drug-induced, autoimmune, and alcoholic hepatitis are known. Chronic hepatic failure is a condition in which a patient who already has a hepatic disorder, such as hepatic cirrhosis, comes into hepatic failure for some cause. The incidences of clinical symptoms, such as general malaise, abdominal distension, anorexia, and cutaneous pruritus, are high in hepatic cirrhosis. A bleeding tendency, ascites, edema, and hematemesis/melena indicate that hepatic cirrhosis has become more serious. These symptoms progress, leading to occurrence of so-called hepatic failure symptoms, such as hepatic encephalitis, a bleeding tendency, ascites, and jaundice.

Among these symptoms, a bleeding tendency is said to be one of the most troublesome symptoms for physicians in the departments of gastrointestinal medicine and surgery who deal with patients with hepatic cirrhosis. Although there remains a lot to be elucidated for the causes of a bleeding tendency, abnormality in blood coagulation and fibrinolysis, and a decrease in platelet count are possible. Hepatocytes synthesize and secrete numerous proteins involved in blood coagulation/fibrinolysis, and, at the same time, the hepatic reticuloendothelial system is able to process and eliminate an activated coagulation factor, a fibrinolysis activator enzyme (plasminogen activator), and the like. In hepatic diseases, depending on the severity, disordered syntheses of coagulation and fibrinolysis factors and inhibitors are observed, as well as a decrease in clearance of plasminogen activator, which indicates a condition of disseminated intravascular coagulation (DIC) syndrome or its preparative state. A decrease in platelet count is, on the other hand, closely related to the progress of pathological conditions from chronic hepatitis to hepatic cirrhosis, and further to decompensated hepatic cirrhosis. The causes of a decrease in platelet count vary widely, and an increase in spleen function, a decrease in platelet production due to megakaryocyte maturation disorder in the bone marrow, and shortened platelet life due to abnormality of platelets themselves have been mainly mentioned so far. There remains a lot to be elucidated to determine the mechanism, however.

While one third of circulating platelets pool in the spleen in healthy individuals, increased portal blood pressure associated with hepatic cirrhosis causes splenomegaly to increase the number of platelets pooled in the spleen, leading to a decrease in the number of platelets in peripheral blood in patients with hepatic cirrhosis. In addition, the platelet count in patients with hepatic cirrhosis who underwent splenectomy to avoid complications due to increased portal blood pressure recovers to a level similar to that in patients with chronic hepatitis. Splenomegaly is thus undoubtedly one of the causes, however, the number of platelets in peripheral blood does not highly correlate with the severity of splenomegaly.

On the other hand, thrombopoietin (sometimes abbreviated as “TPO” hereinafter) was cloned as a major cytokine involved in the platelet production system in 1994, and hepatocytes, pelvic stroma cells, the kidneys, and the like were identified as its production sites. The relationship between chronic hepatic diseases and TPO production thus attracted attention, but most reports state that blood TPO levels in patients with chronic hepatitis or hepatic cirrhosis did not differ from that in healthy individuals. A comparison between patients with hepatic cirrhosis who underwent splenectomy and those with chronic hepatitis shows no difference in the platelet count or blood TPO level. The blood TPO level is almost constant and is not considered to be related with the number of platelets in peripheral blood in hepatic cirrhosis, unless there is a condition in which megakaryocyte and platelet productions in the bone marrow remarkably decrease. A change in platelet count due to increased coagulation/fibrinolysis, involvement of autoantibody in platelet membrane glycoprotein, qualitative platelet abnormality, and the like can also be mentioned as one of the causes in some patients.

Since hepatic failure with hepatic cirrhosis as an underlying disease exhibits various pathological features and is highly probably repeated, routine definite follow-up and preventive measures are essential in addition to grasping pathological conditions and administering appropriate symptomatic therapies. A supplement of coagulation/fibrinolysis factors and correction of coagulation/fibrinolysis are said to be important as well as commonly used hemostatic agents for the management of a bleeding tendency in patients with hepatic failure. Fresh frozen plasma is administered as a replacement therapy, however, there is a concern about a risk of viral infection and antibody production. Gabexate mesylate is used for the correction of abnormal blood coagulation when hepatic failure is severe and complication of DIC is expected. However, gabexate mesylate is less satisfactory as a drug, since attention must be paid upon administration when vascular phlebitis, ulcer, necrosis, and the like occurs at the administration site due to cytotoxicity at a high dose. Serious adverse drug reactions, such as shock, anaphylactic shock, and a decrease in leukocyte count are known, but it has no direct effect on a decease in platelet count. Heparin is also used to correct abnormal blood coagulation when complication of DIC exists, however, its anti-thrombin effect depends on AT-III produced in the liver and thus concomitant administration of AT-III preparation is required, and adverse drug reactions, such as an increased bleeding tendency due to prolongation of drug effects, are known. For acute hepatic failure, plasmapheresis is performed in addition to symptomatic therapies. Plasmapheresis also plays a role of supplementing hepatic functions by replacing albumin and coagulation factors and the like while taking part in detoxication and excretion, the functions of the liver, by exchanging most of plasma components including low to high molecular hazardous substances in the patient blood. Plasmapheresis is thus commonly used for fluminant hepatitis and considered to be indicated for a part of hepatic cirrhosis. Plasmapheresis suffers from problems, such as a high medical cost, necessity of hospitalization, a risk of viral infection, systemic infection, and bleeding at a catheterization site; however, since plasmapheresis is generally conducted several times continuously via an indwelling central venous catheter. As described above, although several therapeutic methods are available for normalization of coagulation/fibrinolysis functions against the bleeding tendency in patients with hepatic failure, no therapeutic method effective for a decrease in platelet count with few adverse drug reactions has been established.

In addition, although interferon (IFN) has been recognized as the only one currently available therapeutic agent that improves viremia in chronic hepatitis C and employed widely, it is known that the number of platelets gradually decreases with time following its administration in most patients. IFN must be thus decreased in dose or discontinued in some patients even when continuation of the therapy is desired. The decrease in platelet count is therefore a clinically significant problem. When IFN is administered to patients with hepatic cirrhosis, close attention must be paid, since these patients often exhibit a low platelet count of 100,000/μL or lower even before the start of administration. No effective therapeutic method has been established for the adverse drug reaction to interferon.

On the other hand, it has been reported that a plasma concentration of von Willebrand factor (sometimes abbreviated as “vWF” hereinafter) that plays an important role in formation of a platelet thrombus increases in hepatic diseases. For example, a vWF level in patients with biliary hepatic cirrhosis was found to be about 2 times higher than that in healthy individuals, and a similar increase was observed for other hepatic diseases (Int J Microcirc Clin Exp, Vol. 15, p. 75, 1995). A significant correlation was observed between disease progress of hepatic cirrhosis and a plasma vWF level (Hepatology, vol. 23, p. 1377, 1996). The vWF level was 92±20% in healthy individuals, whereas it was elevated to 367±185% in patients with hepatic cirrhosis (J Hepatol, Vol. 30, p. 451, 1999). Furthermore, patients with cirrhosis relating to hepatitis B whose platelet count remarkably decreased were reported to exhibit a plasma vWF level elevated to 506%, resistance to plasmapheresis, and poor prognosis (Ann Hematol, Vol. 80, p. 496, 2001).
vWF is a glycoprotein with a high molecular weight and a multimer structure, synthesized in vascular endothelial cells as well as megakaryocytes, and released into circulating blood. At the same time, vWF is immediately degraded by an enzyme in blood and thereby circulates with its normal multimer size retained. vWF is also known as a marker of vascular injury, since it is released from endothelial cells into the blood by cytokines, shear stress stimulation, or artificial destruction. vWF, in addition to its role as a carrier protein to stabilize coagulation factor VIII, serves as an adhesion protein that binds to glycoprotein Ib (sometimes abbreviated as “GPIb” hereinafter) on the platelet membrane to cause adhesion of platelets to subendothelial tissue in the blood vessels and formation of platelet aggregate in the environment with a high shear stress that occurs in stenosed blood vessels or microcirculation.
vWF consists of subunits having a molecular weight of 260 kD (2,050 amino acid residues), having three consecutive A domains composed of about 200 amino acids each (A1, A2, and A3 are present in this order at 509 to 1111 from the N-terminal). A1 domain (509 to 712), like A3, takes a loop structure via a disulfide bond and is also called A1 loop.

It has recently been elucidated that vWF is closely involved in the pathology of thrombotic thrombocytopenic purpura (sometimes abbreviated as “TTP” hereinafter). TTP is a disease having five classical signs, thrombocytopenia, hemolytic anemia accompanied by erythrocyte destruction due to microangiopathy, labile neuropsychiatric symptoms, fever, and renal disorder, and caused by various factors, such as infection, pregnancy, cancer, organ transplantation, and collagen disease. It is considered that, in TTP patients, the vWF-specific degradation enzyme activity is reduced and thereby vWF having an abnormally high molecular weight multimer is released in the plasma to induce vWF-related thrombus formation in the microvascular vessels and platelets are consumed, leading to a decrease in the number of platelets in the peripheral blood.

More importantly, the vWF-specific cleaving enzyme was cloned and shown to be produced mainly in the liver. Actually, it has been shown that activity of the vWF-specific cleaving enzyme severely decreases with a decrease in platelet count in patients with hepatic cirrhosis caused by congenital biliary atresia and that the activity of the enzyme returns to normal by liver transplantation. A decrease in vWF-specific cleaving enzyme activity was observed together with an increase in vWF antigen level in the plasma of patients with hepatic cirrhosis (Blood, Vol. 98, p. 2730, 2001).

In addition, it is also known that a part of patients with von Willebrand disease (sometimes abbreviated as “vWD” hereinafter) attributable to quantitative or qualitative abnormality of vWF have difficulty in hemostasis associated with a decrease in platelet count. In general, vWD is a congenial abnormality in hemostasis with a bleeding tendency in the skin and mucous membrane as a major sign and attributable to a quantitative decrease (type 1), defect (type 3), or a qualitative abnormality (type 2) of vWF. The incidence is 1.0 to 9.3 in 100,000 persons in the U.S. and Europe, while it is about 0.56 in Japan, but the incidence seems to be increased with recognition of this disease.

Among these three disease types of vWD, in type 2B, binding to GPIb is increased due to abnormality in the GPIb binding domain of vWF. In patients with vWD called platelet-type vWD, on the contrary, binding to vWF is increased due to abnormality in the vWF binding domain of GPIb. It is suggested for both disease types that a platelet thrombus is formed in the body, the vWF high molecular weight multimers are consumed, and the number of platelets decreases, which results in difficulty in hemostasis.

Therapeutic methods for the above described type 2B vWD and platelet-type vWD are characterized as compared to those for other types in that desmopressin (1-desamino-8-D-arginine vasopressin, DDAVP, brand name “Desmopressin Injection”), which is the first choice drug for type 1 vWD and type 3 vWD, is contraindicated. Upon massive bleeding or surgery, human FVIII/vWF preparation is generally administered once or twice daily for 1 to 2 days until hemostasis is achieved. Since the amount of vWF itself is similar to that in healthy individuals as compared to vWD with a quantitative decrease in vWF (type 1) or defect of vWF (type 3), there are many problems. For example, the amount required to correct the function of vWF is naturally higher due to the antagonistic relation with vWF preparation administered, and other effective therapeutic methods are expected.

### SUMMARY OF THE INVENTION

The present invention has been achieved in consideration of the above described present situations, and an object of the present invention is to provide a drug for the treatment of thrombocytopenia caused by hepatic failure, such as hepatic cirrhosis, viral hepatitis, drug-induced hepatitis, autoimmune hepatitis, alcoholic hepatitis, or fluminant hepatitis, or type 2B or platelet-type von Willebrand disease, preferably a drug with fewer side effects.

The present inventors eagerly conducted research to solve the above described problems and have conceived, as a result, the possibility that a vWF multimer having an abnormally high molecular weight caused by a decrease in production of vWF-specific cleaving enzyme causes a decrease in platelet count through a mechanism similar to that of TTP in a disease in which organic and functional disorders are observed in the liver, especially in the condition in which hepatocytes are not produced, particularly such as decompensated hepatic cirrhosis, and the possibility that vWF multimer having an abnormally high molecular weight plays a role in a decrease in platelet count in multiple organ failure caused by hepatic failure, which has conventionally been treated as DIC. The present inventors also considered that the substantial problem of a decrease in platelet count and abnormal hemostasis in hepatic failure, and type 2B and platelet-type vWD is a spontaneous onset of platelet thrombus formation in the body due to increased binding between vWF in the plasma and glycoprotein GPIb on the platelet membrane and have conceived the possibility that a drug that inhibits binding between vWF and GPIb can improve a decrease in platelet count, and as a result, redress abnormal hemostasis. The inventors found that inhibition of increased binding between vWF in the plasma and glycoprotein on the platelet membrane, that is, inhibition of platelet aggregate formation in which abnormally high molecular multimer of vWF is involved can redress difficulty in hemostasis through an increase in single platelets and completed the present invention. In addition, the present invention is not limited to the above described syndromes and can be applied to all patients in whom the vWF-specific degradation enzyme activity is reduced for some reasons.

Namely, the summary of the present invention is as follows:

It is an object of the present invention to provide a pharmaceutical composition for the treatment of thrombocytopenia caused by at least one of hepatic failure selected from the group consisting of hepatic cirrhosis, viral hepatitis, drug-induced hepatitis, autoimmune hepatitis, alcoholic hepatitis, and fulminant hepatitis, comprising as an active ingredient a substance that inhibits binding between glycoprotein Ib and von Willebrand factor.

It is a further object of the present invention to provide a pharmaceutical composition for the treatment of type 2B or platelet-type von Willebrand disease comprising as an active ingredient a substance that inhibits binding between glycoprotein Ib and von Willebrand factor.

It is a further object of the present invention to provide a pharmaceutical composition for the treatment of thrombocytopenia caused by administration of interferon for the treatment of viral hepatitis, comprising as an active ingredient a substance that inhibits binding between glycoprotein Ib and von Willebrand factor.

It is a further object of the present invention to provide the pharmaceutical composition as recited above, in which the substance that inhibits binding between glycoprotein Ib and von Willebrand factor is a substance that binds to glycoprotein Ib or von Willebrand factor.

It is a further object of the present invention to provide the pharmaceutical composition as recited above, in which the substance that inhibits binding between glycoprotein Ib and von Willebrand factor is an antibody that binds to glycoprotein Ib or von Willebrand factor.

It is a further object of the present invention to provide the pharmaceutical composition as recited above, in which the substance that inhibits binding between glycoprotein Ib and von Willebrand factor is a monoclonal antibody that binds to glycoprotein Ib or von Willebrand factor, or a chimeric antibody, humanized antibody, complete human antibody or a fragment thereof derived from the monoclonal antibody that inhibits binding between glycoprotein Ib and von Willebrand factor.

It is a further object of the present invention to provide the pharmaceutical composition as recited above, in which the substance that inhibits binding between glycoprotein Ib and von Willebrand factor is a monoclonal antibody whose epitope is located at the GPIb binding site of von Willebrand factor or the vicinity thereof.

It is a further object of the present invention to provide the pharmaceutical composition as recited above, in which the substance that inhibits binding between glycoprotein Ib and von Willebrand factor is a monoclonal antibody whose epitope is located in Al domain of von Willebrand factor.

It is a further object of the present invention to provide the pharmaceutical composition as recited above, in which the substance that inhibits binding between glycoprotein Ib and von Willebrand factor comprises monoclonal antibodies produced by hybridomas deposited with Accession Nos. FERM BP-5247, FERM BP-5248, FERM BP-5249, and/or FERM BP-5250.

### BRIEF DESCRIPTION OF THE DRAWING

FIG. 1 shows the effect of AJW200 on the platelet count when administered at a dose of 0.1 mg/kg for 7 consecutive days to a rat hepatic disorder model prepared by 3-week intraperitoneal administration of DMN 3 times a week. (****: P<0.0001 vs Normal, #: P<0.05 vs Control, t-test).

### DESCRIPTION OF THE PREFERRED EMBODIMENTS

The present invention will be described in more detail below.

The pharmaceutical composition for the treatment of thrombocytopenia according the present invention contains as an active ingredient a substance that inhibits binding between GPIb and vWF. The substance that inhibits binding between GPIb and vWF includes substances that bind to GPIb or vWF and thereby inhibit binding between GPIb and vWF. The present inventors evaluated the therapeutic effect of the humanized antibody (AJW200) of the monoclonal antibody that is produced by hybridoma AJvW-2 and is reactive to vWF using a rat hepatic disorder model as shown in the examples described below, based on the finding that the substantial problem in a decrease in platelet count in at least one hepatic failure selected from hepatic cirrhosis, viral, drug-induced, autoimmune, or alcoholic hepatitis, or fluminant hepatitis, abnormal hemostasis, and type 2B and platelet-type vWD is spontaneous onset of platelet thrombus formation in the body due to increased binding between vWF in the plasma and glycoprotein GPIb on the platelet membrane In other words, the finding that a decrease in platelet count or abnormal hemostasis can be redressed by inhibition of platelet aggregate formation by inhibiting increased binding between vWF in the plasma and glycoprotein GPIb on the platelet membrane. It was found that AJW200 has an effect of improving a decrease in platelet count associated with the progress of disease, and completed the present invention.

Specific examples of the substance having an effect of inhibiting binding between GPIb and vWF include compounds that exhibit an inhibitory effect on platelet aggregation in which binding between GPIb and vWF is involved. As compounds that inhibit platelet aggregation induced by an antibiotic, ristocetin, (M. A. Howard, B. G. Firkin, Thromb. Haemostasis, 26, 362-369 (1971)) and a protein derived from snake venom, botrocetin, (M. S. Read et al., Proc. Natl. Acad. Sci. U.S.A., 75, 4514-4518 (1978)), aurin tricarboxylic acid (M. D. Phillips et al., Blood, 72, 1989-1903 (1988); Golino P, Ragni M, Cirillo P, Pascucci I, Ezekowitz M D, Pawashe A, Scognamiglio A, Pace L, Guarino A, Chiariello M. Aurintricarboxylic acid reduces platelet deposition in stenosed and endothelially injured rabbit carotid arteries more effectively than other antiplatelet interventions. Thromb Haemost. 1995 September; 74(3): 974-9) and dye substances, such as aromatic amidino compounds (J. D. Geratz, et al., Thromb. Haemostasis, 39, 411-425, (1978)), as well as partial fragment peptides of vWF and GPIb (Y. Fujimura et al., J. Biol. Chem., 261, 381-385 (1986), K. Titani et al., Proc. Natl. Acad. Sci. U.S.A., 84, 5610-5614 (1987)) are known. The partial fragment peptides of vWF or GPIb preferably contain the sequence of the respective binding region, and further they may be partially mutated to increase binding ability or fused protein with a partial peptide, for example, Fc region of immunoglobulin protein subjected to mutation to prevent binding of a complement and/or Fc receptor. In addition, the partial fragment peptides may be modified to increase their half-lives in blood and/or decrease their antigenicity, and the like.

Specific examples of the partial fragment of vWF include fragments containing A1 loop (Dardik R, Varon D, Eskaraev R, Tamarin I., Inbal A, The recombinant fragment of von Willebrand factor AR545C inhibits platelet binding to thrombin and platelet adhesion to thrombin-treated endothelial cells. Br J Haematol. 2000 June; 109 (3): 512-8; MaGhie A I, McNatt J, Ezov N, Cui K, Mower L K, Hagay Y, Buja L M, Garfinkel L I, Gorecki M, Willerson J T. Abolition of cyclic flow variations in stenosed, endothelium-injured coronary arteries in nonhuman primates with a peptide fragment (VCL) derived from human plasma von Willebrand factor-glycoprotein Ib binding domain. Circulation. 1994 December; 90(6): 2976-81).

Peptides from snake venoms that have a similar platelet aggregation inhibition activity have been reported, and International Publication WO9208472 describes peptides consisting of different double-strands having a molecular weight of about 25 kD and having a highly homologous amino acid sequence at least for the N-terminal side from Crotalus horridus horridus and Cerastes cerastes. A peptide that inhibits platelet aggregation obtained from Echis carinatus reported by Peng et al. (M. Peng et al., Blood, 81, 2321-2328 (1993)) also is similar to the above described peptides in terms of in vitro activity, molecular weight, and the like. Snake venoms are described in detail in the following articles: Yen C H, Chang M C, Peng H C, and Huang T F. Pharmacological characterization and antithrombotic effect of agkistin, a platelet glycoprotein Ib antagonist. Br J. Pharmacol. 2001 February; 132(4): 843-50; Chang M C, Lin H K, Peng H C, Huang T F. Antithrombotic effect of crotalin, a platelet membrane glycoprotein Ib antagonist from venom of Crotalus atrox. Blood. 1998 Mar. 1; 91 (5): 1582-9; Kawasaki T, Taniuchi Y, Hisamichi N, Fujimura Y, Suzuki M, Titani K, Sakai Y, Kaku S, Satoh N, Takenaka T, et al. Tokaracetin, a new platelet antagonist that binds to platelet glycoprotein ib and inhibits von Willebrand factor-dependent shear-induced platelet aggregation. Biochem J. 1995 Jun. 15; 308 (Pt 3): 947-53; Peng M, Lu W, Beviglia L, Niewiarowski S, Kirby E P. Echicetin: a snake venom protein that inhibits binding of von Willebrand factor and alboaggregins to platelet glycoprotein Ib. Blood. 1993 May 1; 81(9): 2321-8.

Single-stranded peptides obtained from multimer peptides derived from snake venoms that have an activity of inhibiting binding between vWF and platelets can be preferably employed in the present invention (WO95/08573, WO 00/59926). Specifically, such single-stranded peptides include a single-stranded peptide AS1051 derived from snake venom of Crotalus horridus horridus. The peptide is known to bind specifically to GPIb (WO95/08573). Such single-stranded peptides are excellent in that they do not cause a decrease in platelet count generally observed for snake venoms.

In addition to the above described substances, substances that bind to GPIb include antibodies that bind to GPIb (Cauwenberghs N, Ajzenberg N, Vauterin S, Hoylaerts M F, Declerck P J, Baruch D, Deckmyn H. Characterization of murine anti-glycoprotein Ib monoclonal antibodies that differentiate between shear-induced and ristocetin/botrocetin-induced glycoprotein Ib-von Willebrand factor interaction. Haemostasis. 2000 May-Jun; 30(3): 139-48; Cauwenberghs N, Meiring M, Vauterin S, van Wyk V, Lamprecht S, Roodt JP, Novak L, Harsfalvi J, Deckmyn H, Kotze H F. Antithrombotic effect of platelet glycoprotein Ib-blocking monoclonal antibody Fab fragments in nonhuman primates. Arterioscler Thromb Vasc Biol. 2000 May; 20(5): 1347-53). According to the present invention, among these anti-GPIb antibodies, antibodies that bind to the binding site of GPIb to vWF or the vicinity thereof to inhibit binding between GPIb and vWF are preferable. Specifically, a mouse monoclonal antibody against human GPIb called 6B4 is preferable (Arterioscler Thromb Vasc Biol. 2000 May; 20(5): 1347-53).

In addition, substances that bind to vWF include antibodies that bind to vWF. According to the present invention, among the anti-vWF antibodies, antibodies that bind to the binding site of vWF to GPIb or the vicinity thereof to inhibit binding between vWF and GPIb are preferable.

Anti-GPIb antibodies or anti-vWF antibodies employed in the present invention may be polyclonal antibodies or monoclonal antibodies, as far as they inhibit binding between GPIb and vWF. Antibodies may be derivatives from monoclonal antibodies, as far as they bind to GPIb or vWF to inhibit binding between GPIb and vWF.

Anti-GPIb polyclonal antibodies or anti-vWF polyclonal antibodies can be produced according to the conventional method by immunizing mammals used for the production of antibodies, such as mice, rats, rabbis, goats, and sheep, with GPIb or vWF and separating an immunoglobulin fraction from the serum. Anti-GPIb monoclonal antibodies or anti-vWF monoclonal antibodies can be produced according to the method of Kohler and Milstein (Nature, pp. 495-492, 1975) by preparing the respective hybridomas that produces the respective monoclonal antibodies and purifying the antibodies from the supernatant of culture of the hybridomas obtained.

The derivatives of the above described antibodies include chimeric antibodies, humanized antibodies, complete human antibodies, or fragments thereof. Chimera antibodies are, for example, those in which to a constant region of an antibody of a certain animal, and a variable region derived from antibody of another animal are linked. Humanized antibodies are those in which only the gene sequence of the complementarity determining region (CDR) of a nonhuman antibody is introduced into a human antibody gene. According to the present invention, humanized antibodies are preferable, since they are substantially nonantigenic in humans. Humanized antibodies can be produced, for example, by expressing in host cells heavy-chain and light-chain genes obtained by introducing a complementarity determining region of a mouse monoclonal antibody into a framework region of a variable region of an antibody derived from human myeloma by site-specific mutation (Co M S, Yano S, Hsu R K, Landolfi N F, Vasquez M, Cole M, Tso J T, Bringman T, Laird W, Hudson D, et al., A humanized antibody specific for the platelet integrin gpIIb/IIIa. J Immunol 1994 Mar. 15; 152 (6): 2968-76). A DNA sequence encoding a human constant region can be isolated from various human cells, preferably from immortalized B cells according to known methods (Kabat, E., et al., U.S. Department of Health and Human Services, (1987), WO 87/02671). A DNA sequence encoding the variable region of anti-GPIb antibody or anti-vWF antibody can be isolated from cells producing the respective antibodies. A host in which a chimeric gene is expressed can be obtained from numerous suppliers, such as American Type Culture Collection (Catalogue of Cell Lines and Hybridomas, Fifth edition (1985) Rockville, Md., U.S.A.).

The antibody fragments include F(ab′)₂, Fab′, Fab, and Fv. Techniques such as bifunctional hybrid antibody (Lanzavecchia et al., Eur. J. Imunol. 17, 105 (1987)) and a single-strand (Huston et al., Proc. Natl. Acad. Sci. U.S.A., 85, 5879-5883 (1988) and Bird et al., Science 242, 423-426 (1988)), can also be applied to the present invention.

Monoclonal antibodies whose epitope is located at the GPIb binding site of vWF or the vicinity thereof are preferable as anti-vWF antibodies. Specifically, these anti-vWF antibodies include a monoclonal antibody whose epitope is located in the A1 domain of vWF. More specifically, monoclonal antibodies produced by hybridomas AJvW-1, AJvW-2, AJvW-3, and AJvW-4 can be preferably mentioned as anti-vWF antibodies whose epitope is located at the GPIb binding site or the vicinity thereof (WO96/17078). These hybridomas were deposited at National Institute of Bioscience and Human-Technology, Agency of Industrial Science and Technology, the Ministry of International Trade and Industry (presently, National Institute of Advanced Industrial Science and Technology, Independent Administrative Institution, International Patent Organism Depository, Tsukuba Central 8, 1-1-1 Higashi, Tsukuba, Ibaraki, 305-8566, Japan) with Accession Numbers of FERM P-14486, FERM P-14487, FERM P-14488, and FERM P-14489, respectively, in this order on Aug. 24, 1994, and then converted to the International Organism Depository according to the Budapest Treaty on the International Recognition of the Deposit of Microorganisms for the Purposes of Patent Procedure and deposited with Accession Numbers of FERM BP-5247, FERM BP-5248, FERM BP-5249, and FERM BP-5250, respectively, in this order on Sep. 29, 1995. A monoclonal antibody produced by AJvW-2 is described in detail in references (Br J Pharmacol, Vol. 122, p. 165, 1997 and Thromb Haemost, Vol. 79, p. 202, 1998) and a patent (WO96/17078).

Among the above described antibodies, monoclonal antibodies produced by AJvW-2 and AJvW-4 have the following properties:
(a) They have reactivity to human von Willebrand factor.
(b) They inhibit RIPA (ristocetin-induced platelet aggregation reaction), BIPA (botrocetin-induced platelet aggregation reaction), and SIPA (high shear-induced platelet aggregation reaction) of human platelets.
(c) They inhibit RIPA (ristocetin-induced platelet aggregation reaction) and BIPA (botrocetin-induced platelet aggregation reaction) of guinea pig platelets.
(d) They exhibit a strong anti-thrombotic effect, but they do not induce bleeding, in vivo in guinea pigs.

Monoclonal antibodies produced by AJvW-1 and AJvW-3 have the following properties:
(A) They have reaction specificity to human von Willebrand factor.
(B) They inhibit RIPA (ristocetin-induced platelet aggregation reaction), BIPA (botrocetin-induced platelet aggregation reaction), and SIPA (high shear-induced platelet aggregation reaction) of human platelets.
(C) They do not react with rat, guinea pig, or rabbit von Willebrand factor.

It was shown that a monoclonal antibody produced by AJvW-2 has an epitope on A1 loop of vWF by the findings that VCL, an A1 loop fragment, inhibits competitively binding of the antibody to vWF and that VCL that has been reduced and alkylated to lose a loop structure does not inhibit competitively the binding (Yamamoto H, Vreys I, Stassen J M, Yoshimoto R, Vermylen J, Hoylaerts M F. Antagonism of vWF inhibits both injury induced arterial and venous thrombosis in the hamster. Thromb Haemost. 1998; 79: 202-210). As described in WO96/17078, it has been shown that monoclonal antibodies produced by AJvW-1, AJvW-3, and AJvW-4 inhibit competitive binding of a monoclonal antibody produced by AJvW-2 to vWF, respectively. These three antibodies are therefore considered to have their binding sites to vWF in the vicinity of the binding site of AJvW-2 on A1 loop.

The most preferable antibody in the present invention is a monoclonal antibody produced by AJvW-2 or a humanized antibody thereof. The humanized antibody and its production method are disclosed in detail in International Publication WO 00/10601. The humanized antibody (referred to as AJW200) obtained by the method described in the examples in the International Publication was employed in the examples below.

The drug for the treatment of thrombocytopenia according to the present invention is characterized in that it contains as an active ingredient a substance that inhibits binding between GPIb and vWF as described above. As dosage forms of the drug for the treatment of thrombocytopenia according to the present invention, compositions in the form of an injection, sublingual, transcutaneous adhesive patch, tablet, capsule, fine granule, syrup, suppository, ointment, eye drop, and the like may be mentioned.

The content of the substance that inhibits binding between GPIb and vWF in the drug according to the present invention is preferably 0.01 to 100 parts by weight with respect to 100 parts by weight of the drug. The substance may be used alone or as a mixture of any two or more substances.

The drug according to the present invention may be formulated with pharmaceutically acceptable exipients and extenders, for example, dextrin, lactose, potato starch, calcium carbonate, sodium alginate, and the like, depending on dosage form. The drug according to the present invention may be in any form of liquid, powder, capsule, and granule. In case of injection, distilled water for injection, physiological saline, phosphate buffer, linger solution, and the like are used as a solvent, and a dispersant may be added thereto. Antithrombotic ingredients other than the substance that inhibits binding between GPIb and vWF may be used in combination.

As patients for whom the drug according to the present invention is indicated, patients with hepatic disorder and patients with thrombocytopenia caused by hepatic failure, such as hepatic cirrhosis, viral hepatitis, drug-induced hepatitis, autoimmune hepatitis, alcoholic hepatitis or fulminant hepatitis, and patients with type 2B and platelet-type von Willebrand disease may be mentioned, but the drug according to the present invention can be indicated to patients in whom the binding between vWD and GPIb is increased to cause a decrease in platelet count and thereby a bleeding tendency is exhibited and patients in whom liver function is deteriorated for some reasons and vWF-specific degradation enzyme activity falls and thereby a bleeding tendency is exhibited. The drug according to the present invention is administered to redress a bleeding tendency. The drug according to the present invention may be administered to improve thrombocytopenia caused by administration of interferon for the treatment of viral hepatitis as well. In addition, the drug according to the present invention may be administered in combination with interferon for the prevention of thrombocytopenia as side effects to interferon therapy. As administration routes, oral administration, intravenous administration, sublingual absorption, transcutaneous absorption, enteral absorption, eyedrop instillation, and the like may be mentioned. In case of antibody, single intravenous administration may be sufficiently effective, but antibody can be administered several times unless it has an antigenic problem. The dose of the drug according to the present invention is not limited, as far as its therapeutic effect is produced, but it is administered at a dose in the range of 0.1 μg/kg to 1000 mg/kg for adults. The dose is preferably in the range of 10 μg/kg to 30 mg/kg.

In the present invention, as thrombocytopenia, thrombocytopenia caused by hepatic failure, such as hepatic cirrhosis, viral hepatitis, drug-induced hepatitis, autoimmune hepatitis, alcoholic hepatitis, or fulminant hepatitis, thrombocytopenia associated with increased thrombus formation in the vascular lumen such as type 2B or platelet-type von Willebrand disease and the like, and thrombocytopenia caused by interferon administration for the treatment of viral hepatitis may be mentioned.

As interferon used for the treatment of viral infection that is the cause of thrombocytopenia for which the drug according to the present invention is indicated, natural interferon α, NAMALWA and BALL-1, interferon β, interferon γ-n1, and the like are known. In addition, gene recombinant interferons α-2a and α-2b, and interferon alfacon-1 and interferon γ-1a, and the like are known. As viral hepatitis, chronic active hepatitis B, chronic active hepatitis C, and the like may be mentioned, but all other types of hepatitis caused by virus for which interferon therapy is indicated are included.

### EXAMPLES

The present invention will be further described in detail by the following non-limiting examples. In Example 1 below, humanized anti-von Willebrand factor monoclonal antibody AJW 200 produced by hybridoma AJvW-2 was used as an inhibitor of binding between glycoprotein Ib and von Willebrand factor to specifically explain that a substance that inhibits binding between glycoprotein Ib and von Willebrand factor is an effective therapeutic agent for thrombocytopenia to increase the number of free platelets in blood. The humanized antibody and its production method are fully disclosed in International Publication WO 00/10601.

### Example 1

### Efficacy Evaluation in Rat Hepatic Disorder Model (1)

N-nitrosodimethylamine (DMA) was administered intraperitoneally to male SD rats at a dose of 10 mg/kg 3 times a week for 3 weeks to prepare a rat hepatic disorder model. In Week 3, AJW200 was administered from the caudal vein at 0.1 mg/kg once daily for 7 days. PBS that was a solvent for AJW200 was administered in a similar manner to the disease control group. The group constitution is shown below.
1. Normal control group (n=10)
2. Disease control group (n=8)
3. AJW200 administration group (n=8)

Blood was collected one day after the final administration and hematological parameters were measured.

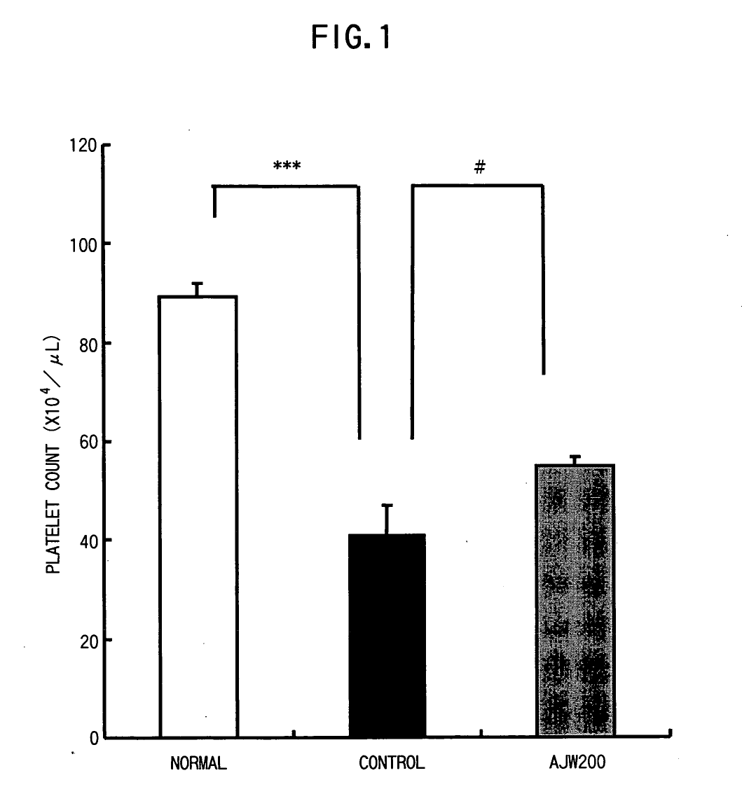

The results are shown in FIG. 1. The platelet count decreased significantly in the disease control group as compared to the normal control group. On the contrary, the platelet count increased significantly in the AJW200 administration group (administration for 7 days) as compared to the disease control group (FIG. 1).

### Example 2

### Efficacy Evaluation in Rat Hepatic Disorder Model (2)

N-nitrosodimethylamine (DMA) is administered intraperitoneally to male SD rats at a dose of 10 mg/kg 3 times a week for 3 weeks to prepare a rat hepatic disorder model. In Week 3, glycoprotein Ib partial peptide at a dose of 0.1 to 1000 μg/kg, preferably glycoprotein Ib partial peptide at a dose of 1 to 100 μg/kg is administered from the caudal vein once daily for 7 days. A solvent for the glycoprotein Ib partial peptide is administered in a similar manner to the disease control group. Blood is collected one day after the final administration and hematological parameters are measured. This method allows confirmation of a significant increase in platelet count by glycoprotein Ib partial peptide.

The above-described experimental results show that the substance that inhibits binding between GPIb and vWF has a therapeutic effect on thrombocytopenia by an effect of inhibiting binding between GPIb and vWF in the hepatic disorder model. This result suggested the possibility that the substance that inhibits binding between GPIb and vWF has a therapeutic effect on type 2B and platelet-type von Willebrand disease considered to develop thrombocytopenia via a similar development mechanism, that is, increase of binding between GPIb and vWF.

### Industrial Applicability

The substances that inhibit binding between GPIb and vWF represented by monoclonal antibody AJW200 improve the effect on thrombocytopenia caused by hepatic failure, such as hepatic cirrhosis, viral hepatitis, drug-induced hepatitis, autoimmune hepatitis, alcoholic hepatitis, or fulminant hepatitis, and are also effective for the treatment of type 2B or platelet-type von Willebrand disease. Such an excellent effect of improving thrombocytopenia is also effective for thrombocytopenia caused by interferon administration for the treatment of viral hepatitis.

While the invention has been described in detail with reference to preferred embodiments thereof, it will be apparent to one skilled in the art that various changes can be made, and equivalents employed, without departing from the scope of the invention. Each of the aforementioned documents, including the foreign priority documents, JP 2002-219173, is incorporated by reference herein in its entirety.

1. A method for the treatment of thrombocytopenia caused by at least one hepatic failure selected from the group consisting of hepatic cirrhosis, viral hepatitis, drug-induced hepatitis, autoimmune hepatitis, alcoholic hepatitis, and fulminant hepatitis, comprising administrating as an active ingredient a substance that inhibits binding between glycoprotein Ib and von Willebrand factor to a patient in need thereof.

2. The method according to claim 1, wherein said substance that inhibits binding between glycoprotein Ib and von Willebrand factor is a substance that binds to glycoprotein Ib or von Willebrand factor.

3. The method according to claim 1, wherein said substance that inhibits binding between glycoprotein Ib and von Willebrand factor is an antibody that binds to glycoprotein Ib or von Willebrand factor.

4. The method according to claim 1, wherein said substance that inhibits binding between glycoprotein Ib and von Willebrand factor is a monoclonal antibody that binds to glycoprotein Ib or von Willebrand factor, or a chimeric antibody, humanized antibody, complete human antibody or a fragment thereof derived from the monoclonal antibody that inhibits binding between glycoprotein Ib and von Willebrand factor.

5. The method according to claim 1, wherein said substance that inhibits binding between glycoprotein Ib and von Willebrand factor is a monoclonal antibody whose epitope is located at the GPIb binding site of von Willebrand factor or the vicinity thereof.

6. The method according to claim 1, wherein said substance that inhibits binding between glycoprotein Ib and von Willebrand factor is a monoclonal antibody whose epitope is located in the A1 domain of von Willebrand factor.

7. The method according to claim 1, wherein said substance that inhibits binding between glycoprotein Ib and von Willebrand factor comprises monoclonal antibodies produced by hybridomas deposited with Accession Nos. FERM BP-5247, FERM BP-5248, FERM BP-5249, and/or FERM BP-5250.

8. A method for the treatment of type 2B or platelet-type von Willebrand disease comprising administrating as an active ingredient a substance that inhibits binding between glycoprotein Ib and von Willebrand factor to a patient in need thereof.

9. The method according to claim 8, wherein said substance that inhibits binding between glycoprotein Ib and von Willebrand factor is a substance that binds to glycoprotein Ib or von Willebrand factor.

10. The method according to claim 8, wherein said substance that inhibits binding between glycoprotein Ib and von Willebrand factor is an antibody that binds to glycoprotein Ib or von Willebrand factor.

11. The method according to claim 8, wherein said substance that inhibits binding between glycoprotein Ib and von Willebrand factor is a monoclonal antibody that binds to glycoprotein Ib or von Willebrand factor, or a chimeric antibody, humanized antibody, complete human antibody or a fragment thereof derived from the monoclonal antibody that inhibits binding between glycoprotein Ib and von Willebrand factor.

12. The method according to claim 8, wherein said substance that inhibits binding between glycoprotein Ib and von Willebrand factor is a monoclonal antibody whose epitope is located at the GPIb binding site of von Willebrand factor or the vicinity thereof.

13. The method according to claim 8, wherein said substance that inhibits binding between glycoprotein Ib and von Willebrand factor is a monoclonal antibody whose epitope is located in A1 domain of von Willebrand factor.

14. The method according to claim 8, wherein said substance that inhibits binding between glycoprotein Ib and von Willebrand factor comprises monoclonal antibodies produced by hybridomas deposited with Accession Nos. FERM BP-5247, FERM BP-5248, FERM BP-5249, and/or FERM BP-5250.

15. A method for the treatment of thrombocytopenia caused by administration of interferon for the treatment of viral hepatitis, comprising administrating as an active ingredient a substance that inhibits binding between glycoprotein Ib and von Willebrand factor to a patient in need thereof.

16. The method according to claim 15, wherein said substance that inhibits binding between glycoprotein Ib and von Willebrand factor is a substance that binds to glycoprotein Ib or von Willebrand factor.

17. The method according to claim 15, wherein said substance that inhibits binding between glycoprotein Ib and von Willebrand factor is an antibody that binds to glycoprotein Ib or von Willebrand factor.

18. The method according to claim 15, wherein said substance that inhibits binding between glycoprotein Ib and von Willebrand factor is a monoclonal antibody that binds to glycoprotein Ib or von Willebrand factor or a chimeric antibody, humanized antibody, complete human antibody or a fragment thereof derived from the monoclonal antibody that inhibits binding between glycoprotein Ib and von Willebrand factor.

19. The method according to claim 15, wherein said substance that inhibits binding between glycoprotein Ib and von Willebrand factor is a monoclonal antibody whose epitope is located at the GPIb binding site of von Willebrand factor or the vicinity thereof.

20. The method according to claim 15, wherein said substance that inhibits binding between glycoprotein Ib and von Willebrand factor is a monoclonal antibody whose epitope is located in A1 domain of von Willebrand factor.

21. The method according to claim 15, wherein said substance that inhibits binding between glycoprotein Ib and von Willebrand factor comprises monoclonal antibodies produced by hybridomas deposited with Accession Nos. FERM BP-5247, FERM BP-5248, FERM BP-5249, and/or FERM BP-5250.
